(19) United States
(12) Patent Application Publication
(10) Pub. No.: US 2026/0102797 A1
(43) Pub. Date: Apr. 16, 2026

(54) Biodegrading Restaurant Packaging with Leftover Food

(71) Applicant: University of North Texas at Dallas, Dallas, TX (US)

(72) Inventor: Mohammadmehdi Eslamieh, Plano, TX (US)

(21) Appl. No.: 19/352,616

(22) Filed: Oct. 8, 2025

Related U.S. Application Data
(60) Provisional application No. 63/705,916, filed on Oct. 10, 2024.

(51) Int. Cl. B09B 3/60; A01K 67/33; B09B 101/70; B09B 101/75; B09B 101/77

(52) U.S. Cl. CPC B09B 3/60; A01K 67/33; B09B 2101/70; B09B 2101/75; B09B 2101/77

(57) ABSTRACT

Provided herein are methods for degrading a combination of food stuffs and non-biodegradable food packaging, the method comprising: obtaining a combination of the food stuffs and non-biodegradable food packaging; contacting the combination of food stuffs and non-biodegradable food packaging with a superworm or mealworm; and incubating the combination of food stuffs and non-biodegradable food packaging and the superworm or mealworm under conditions in which the superworm or mealworm degrades both the food stuffs and non-biodegradable food packaging.

### CROSS-REFERENCE TO RELATED APPLICATIONS

This application claims priority to U.S. Provisional Application Ser. No. 63/705,916, filed Oct. 10, 2024, the entire contents of which are incorporated herein by reference.

### TECHNICAL FIELD

The present disclosure relates in general to the field of non-biodegradable packaging, and more particularly, to novel methods of biodegrading restaurant packaging with leftover food.

### STATEMENT OF FEDERALLY FUNDED RESEARCH

None.

### INCORPORATION-BY-REFERENCE OF MATERIALS FILED ON COMPACT DISC

Not applicable.

### BACKGROUND

Without limiting the scope of the disclosure, its background is described in connection with the degradation of food and food packaging.

One such disclosure is found in U.S. Patent Publication No. 20150247018, filed by Yang, et al., entitled “Biodegradation of Petroleum-Based Plastics”. These applicants are said to teach a biological processes for degrading waste plastics and recycled plastics, and more specifically to novel isolated insects capable of degrading petroleum-based plastics, bacterial strains capable of degrading petroleum-based plastics, and microbial consortia including such strains. However, these applicants fail to teach methods for biodegrading mixed compositions that include both the petroleum-based products and other biomaterials.

Another such disclosure is found in U.S. Patent Publication 20070184158, filed by Soane, et al., entitled “Methods and Apparatus for Removal Of Degradation Byproducts And Contaminants From Food Grade Oil”. These applicants are said to teach methods, particles and devices that are suitable for food-grade oil filtration, including particles that comprise an inert porous inner particle and at least a partial coating on the inner particle that comprises a polymer comprising at least one amine, amino or imine group.

One such invention is found in U.S. Pat. No. 5,854,304, issued to Garcia and Gho, entitled, “Degradable/compostable Concentrates, Process for Making Degradable/compostable Packaging Materials and The Products Thereof”. These inventors are said to teach an additive degradable/compostable package that includes a combination of metal carboxylate and an aliphatic poly hydroxy-carboxyl acids, which combination is added to polyolefin resins in the production of specific products. The addition of the additive degradable/compostable package is said to produce a packaging product which is degradable/compostable, and that may further include calcium oxide (CaO) to make the modified polyolefin resin more processable. Further, stabilizers or oxidizers may be added to the additive package.

Despite these advances, a need remains for methods of treating and processing packaging and foodstuffs that that includes non-biodegradable packaging, but can be processed to eliminate some or all of the non-biodegradable packaging.

### SUMMARY

As embodied and broadly described herein, an aspect of the present disclosure relates to a method of degrading a combination of food stuffs and non-biodegradable food packaging, the method comprising: obtaining a combination of the food stuffs and non-biodegradable food packaging; contacting the combination of food stuffs and non-biodegradable food packaging with a superworm or a mealworm; and incubating the combination of food stuffs and non-biodegradable food packaging and the superworm or mealworm under conditions in which the superworm or mealworm degrades both the food stuffs and non-biodegradable food packaging. In one aspect, the food stuffs and non-biodegradable food packaging are both carbon sources and the food stuffs comprise at least one of: plants, meats, and fats. In another aspect, the non-biodegradable food packaging is selected from at least one of: polystyrene (PS), polyethylene (PE), polypropylene (PP), polyvinyl chloride (PVC), polyethylene terephthalate (PET), or polycarbonate (PC). In another aspect, the superworm is Zophobas morio and the mealworm is Tenebrio molitor. In another aspect, the superworm or mealworm are at least one of: incubated with the food stuffs and non-biodegradable food packaging for a period of time that ranges from about 10, 15, 20, 25, 30, 40, 45, 50, 60, 70, 75, 80, 90, 100, 110, 120, 130, 140, or 150 days; gains weight during the incubating step; are grown with the food stuffs and non-biodegradable food packaging for a period of time that ranges from about 2 hours to about 480 hours, from about 2 hours to about 360 hours, from about 2 hours to about 240 hours, from about 2 hours to about 120 hours, from about 2 hours to about 96 hours, from about 2 hours to about 72 hours, from about 2 hours to about 48 hours, or from about 2 hours to about 24 hours; are grown with the food stuffs and non-biodegradable food packaging at a temperature that ranges from about 15° C. to about 45° C.; are grown with the food stuffs and non-biodegradable food packaging at a temperature that ranges from about 25° C. to about 28° C.; are grown with the food stuffs and non-biodegradable food packaging at a moisture content that ranges from about 60% to about 99%; grown with the food stuffs and non-biodegradable food packaging a moisture content that ranges from about 80% to about 90%; or convert the food stuffs and non-biodegradable food packaging to carbon dioxide (CO₂). In another aspect, the method further comprises adding carbohydrate food stuffs to meat- or fat-based foods during the incubating step to increase at least one of a total degradation of the food stuffs and non-biodegradable food packaging, increase a rate of degradation of the food stuffs and non-biodegradable food packaging, increase superworm or mealworm size and/or weight, or decrease superworm or mealworm death. In another aspect, the mealworm is Tenebrio molitor and it biodegrades polystyrene.

As embodied and broadly described herein, an aspect of the present disclosure relates to a method of degrading a combination of food stuffs and non-biodegradable food packaging, the method comprising: obtaining or growing superworms or mealworms and mixing with the food stuffs and non-biodegradable food packaging; and incubating the combination of food stuffs and non-biodegradable food packaging and the superworms or mealworms, wherein the superworms or mealworms degrade both the food stuffs and non-biodegradable food packaging. In one aspect, the food stuffs and non-biodegradable food packaging are both carbon sources and food stuffs comprise at least one of: plants, meats, and fats. In another aspect, the non-biodegradable food packaging is selected from at least one of: polystyrene (PS), polyethylene (PE), polypropylene (PP), polyvinyl chloride (PVC), polyethylene terephthalate (PET), or polycarbonate (PC). In another aspect, the superworm is Zophobas morio and the mealworm is Tenebrio molitor. In another aspect, the superworm or mealworm at least one of: incubated with the food stuffs and non-biodegradable food packaging for a period of time that ranges from about 10, 15, 20, 25, 30, 40, 45, 50, 60, 70, 75, 80, 90, 100, 110, 120, 130, 140, or 150 days; gains weight during the incubating step; are grown with the food stuffs and non-biodegradable food packaging for a period of time that ranges from about 2 hours to about 480 hours, from about 2 hours to about 360 hours, from about 2 hours to about 240 hours, from about 2 hours to about 120 hours, from about 2 hours to about 96 hours, from about 2 hours to about 72 hours, from about 2 hours to about 48 hours, or from about 2 hours to about 24 hours; are grown with the food stuffs and non-biodegradable food packaging at a temperature that ranges from about 15° C. to about 45° C.; are grown with the food stuffs and non-biodegradable food packaging at a temperature that ranges from about 25° C. to about 28° C.; are grown with the food stuffs and non-biodegradable food packaging at a moisture content that ranges from about 60% to about 99%; are grown with the food stuffs and non-biodegradable food packaging a moisture content that ranges from about 80% to about 90%; or convert the food stuffs and non-biodegradable food packaging to carbon dioxide (CO₂). In another aspect, the method further comprises adding carbohydrate food stuffs to meat- or fat-based foods during the incubating step to increase at least one of a total degradation of the food stuffs and non-biodegradable food packaging, increase a rate of degradation of the food stuffs and non-biodegradable food packaging, increase superworm or mealworm size and/or weight, or decrease superworm or mealworm death. In another aspect, the mealworm is Tenebrio molitor and it biodegrades polystyrene.

As embodied and broadly described herein, an aspect of the present disclosure relates to a method of degrading a combination of food stuffs and non-biodegradable food packaging, the method comprising: obtaining a combination of the food stuffs and non-biodegradable food packaging; contacting the combination of food stuffs and non-biodegradable food packaging with Tenebrio molitor; and incubating the combination of food stuffs and non-biodegradable food packaging and the Tenebrio molitor under conditions in which the Tenebrio molitor degrades both the food stuffs and non-biodegradable food packaging. In one aspect, the food stuffs and non-biodegradable food packaging are both carbon sources and the food stuffs comprise at least one of: plants, meats, and fats. In another aspect, the non-biodegradable food packaging is selected from at least one of: polystyrene (PS), polyethylene (PE), polypropylene (PP), polyvinyl chloride (PVC), polyethylene terephthalate (PET), or polycarbonate (PC). In another aspect, the Tenebrio molitor at least one of: incubated with the food stuffs and non-biodegradable food packaging for a period of time that ranges from about 10, 15, 20, 25, 30, 40, 45, 50, 60, 70, 75, 80, 90, 100, 110, 120, 130, 140, or 150 days; gains weight during the incubating step; grown with the food stuffs and non-biodegradable food packaging for a period of time that ranges from about 2 hours to about 480 hours, from about 2 hours to about 360 hours, from about 2 hours to about 240 hours, from about 2 hours to about 120 hours, from about 2 hours to about 96 hours, from about 2 hours to about 72 hours, from about 2 hours to about 48 hours, or from about 2 hours to about 24 hours; grown with the food stuffs and non-biodegradable food packaging at a temperature that ranges from about 15° C. to about 45° C.; grown with the food stuffs and non-biodegradable food packaging at a temperature that ranges from about 25° C. to about 28° C.; grown with the food stuffs and non-biodegradable food packaging at a moisture content that ranges from about 60% to about 99%; grown with the food stuffs and non-biodegradable food packaging a moisture content that ranges from about 80% to about 90%; or convert the food stuffs and non-biodegradable food packaging to carbon dioxide (CO₂). In another aspect, the method further comprises adding carbohydrate food stuffs to meat- or fat-based foods during the incubating step to increase at least one of a total degradation of the food stuffs and non-biodegradable food packaging, increase a rate of degradation of the food stuffs and non-biodegradable food packaging, increase Tenebrio molitor size and/or weight, or decrease Tenebrio molitor death.

### BRIEF DESCRIPTION OF THE DRAWINGS

For a more complete understanding of the features and advantages of the present disclosure, reference is now made to the detailed description of the disclosure along with the accompanying figures and in which:

FIG. 1 is a graph that shows a comparison of food and polystyrene (PS) consumption in superworms between experimental and control groups.

FIG. 2 is a graph that shows a comparison of superworm changes between experimental and control groups.

FIG. 3 is a graph that shows survival/mortality rates for the superworms between experimental and control groups.

FIG. 4 is a graph that shows a comparison of food and PS consumption in mealworms between experimental and control groups.

FIG. 5 is a graph that shows a comparison of Mealworm changes between experimental and control groups.

FIG. 6 is a graph that shows the survival/mortality rates for mealworms between experimental and control groups.

### DETAILED DESCRIPTION

While the making and using of various aspects of the present disclosure are discussed in detail below, it should be appreciated that the present disclosure provides many applicable inventive concepts that can be embodied in a wide variety of specific contexts. The specific aspects discussed herein are merely illustrative of specific ways to make and use the disclosure and do not delimit the scope of the disclosure.

To facilitate the understanding of this disclosure, a number of terms are defined below. Terms defined herein have meanings as commonly understood by a person of ordinary skill in the areas relevant to the present disclosure. Terms such as “a”, “an” and “the” are not intended to refer to only a singular entity, but include the general class of which a specific example may be used for illustration. The terminology herein is used to describe specific aspects of the disclosure, but their usage does not delimit the disclosure, except as outlined in the claims.

Polystyrene, a ubiquitous plastic used in everything from packaging to insulation, is a major contributor to the global plastic waste problem. Despite its widespread use, Polystyrene is highly resistant to biodegradation, leading to its accumulation in landfills. Zophobas morio (superworm) have shown promise in addressing the polystyrene crisis. These beetle larvae have been found to consume and break down polystyrene with the aid of a gut bacterium. The potential of superworms to biodegrade polystyrene offers a glimmer of hope in the fight against plastic pollution. However, research and development are needed to fully explore and harness their capabilities. The inventors show herein that these larvae could biodegrade Polystyrene with different types of leftover foods. Surprisingly, the findings herein demonstrate that superworms are not only capable of biodegrading polystyrene but also possess remarkable adaptability, thriving even in environments with fluctuating food sources. Their impressive resilience to dietary changes underscores their exceptional adaptability.

Polyethylene plastics, known for their durability and versatility, have become a staple in modern consumer products. However, their resistance to degradation poses a significant environmental challenge. Polystyrene (PS), a common type of polyethylene used in packaging and insulation, is particularly problematic due to its widespread use and persistent nature. Landfilling and incineration, the current primary methods for waste disposal, are struggling to cope with the sheer volume of plastic waste. The continued reliance on these methods is unsustainable and contributes to long-term environmental pollution. Addressing the plastic waste crisis requires a multifaceted approach. While reducing plastic consumption is essential, it is unrealistic to eliminate plastic entirely from modern society. Instead, the inventors focused on developing innovative solutions for waste management and promoting circular economy principles to minimize the environmental impact of plastics. While various initiatives concentrate on collecting and recycling plastic waste, recent research has highlighted the potential of natural biological degradation as a sustainable solution. Scientists have isolated a polystyrene-degrading bacterium, Pseudomonas sp., from the gut of superworms (Hong et al., 2020). This bacterium, originally discovered in 1946, appears to have evolved slight variations within the superworm's digestive system. Pseudomonas sp. plays a critical role in superworms' ability to break down polystyrene with enzymes that can degrade the complex hydrocarbons in polystyrene, making it easier for the larvae to digest. While in vivo studies have demonstrated superworms' ability to degrade polystyrene, attempts to replicate this process in vitro (outside of the organism) have been unsuccessful. This suggests that the gut environment plays a crucial role in facilitating polystyrene biodegradation. The mechanisms behind superworms' ability to degrade polystyrene have been recently uncovered. Z. morio and its gut microbiome utilize reactive oxygen species (ROS) to break down polystyrene through oxidative processes (Chen et al., 2023 and Xu et al., 2024). This discovery offers a potential solution for plastic waste and emphasizes the crucial role of bacteria like Pseudomonas in this process. While superworms can consume and partially degrade polystyrene, a diet consisting solely of plastic can negatively impact their gut microbiome diversity and health (Sun et al., 2022). This highlights the importance of balanced feeding strategies to ensure the well-being of superworm populations and the sustainability of their use in plastic biodegradation.

Beyond their ability to biodegrade plastic, superworms offer a promising source of sustainable nutrition. With their high protein content and minimal environmental impact, they are well-suited to meet the growing demand for animal feed or human consumption in certain cultures (Rumbos et al., 2021). Recent EU regulations have paved the way for incorporating insects, including superworms, into aquafeeds and potentially human diets. However, further research is needed to fully understand the nutritional implications of insects that have consumed plastic waste. It is crucial, however, to assess the safety and health implications of consuming products derived from plastic-fed insects (Zielinska et al., 2021). This dual benefit underscores Zophobas morio's potential to contribute to sustainable environmental practices and food security.

### Example 1

To fully exploit the potential of superworms, rigorous research and development are necessary to understand their limitations, constraints, and potential environmental and health risks. This knowledge will enable us to utilize them responsibly and effectively as a sustainable solution for plastic waste. This includes exploring the long-term effects of plastic consumption on superworm health, optimizing biodegradation conditions, and leveraging biotechnology to enhance their efficacy. With continued innovation and careful consideration of ecological and economic factors, superworms could become a pivotal solution in reducing plastic waste, protecting ecosystems, and contributing to sustainable food systems.

Materials and Methods. A commercially available population of superworm larvae (Zophobas morio) was obtained from Carolina Biological Supply (www.carolina.com/beetles/superworm/FAM_144302. pr? srsltid=AfmBOoo_nexFdPMia9kNMEWRMRyFF287Mt0oJtl71DXTEy8vKgaCb-uj; Item #:144302). Upon arrival, the superworms were acclimatized to the laboratory environment for a period of one week to minimize stress-related impacts on the experiment. The superworms were provided with a standardized diet of Bran meal that was obtained from Carolina Biological Supply (www.carolina.com/beetles/bran-meal-25-kg/144320.pr?catId=&mCat=&sCat=;Item#:144320). For this study, the inventors used Dart takeout containers (representing polystyrene) which were obtained from the UNTDallas café where the inventors ordered the other food items. Superworms were housed in plastic containers equipped with mesh screen floors and no lids. These containers were maintained in a controlled laboratory environment with a temperature of 25° C.±1° C. and a relative humidity of 65%±5%. For the experiment, a total of 2,400 superworms were utilized, divided into three experimental groups and one control group. To account for potential variability, each group was further divided into three replicate containers. Each replicate container housed 200 superworms, resulting in a total of 600 superworms per group. This experimental design ensures sufficient statistical power for data analysis. Each group received a weekly allotment of 3 grams of food and 3 grams of polystyrene. The first experimental group was provided with baked potato to represent carbohydrate macromolecules. The second group received baked red meat to represent protein macromolecules. The third group was given baked fat around the meat, simulating lipid macromolecules. The control group received only polystyrene, without any additional food source.

This study lasted for 10 weeks. The weight of the superworms was measured both at the beginning and end of the study. Additionally, the weight of the provided food, polystyrene, leftover food, and remaining polystyrene was measured weekly. The number of dead superworms was counted at week 10 to calculate the mortality rate. In this study, the inventors utilized R for in-depth data analysis to uncover key insights. These analyses provided a robust understanding of the data, enabling us to identify key relationships between variables, uncover significant trends, and draw data-driven conclusions about biodegradation process.

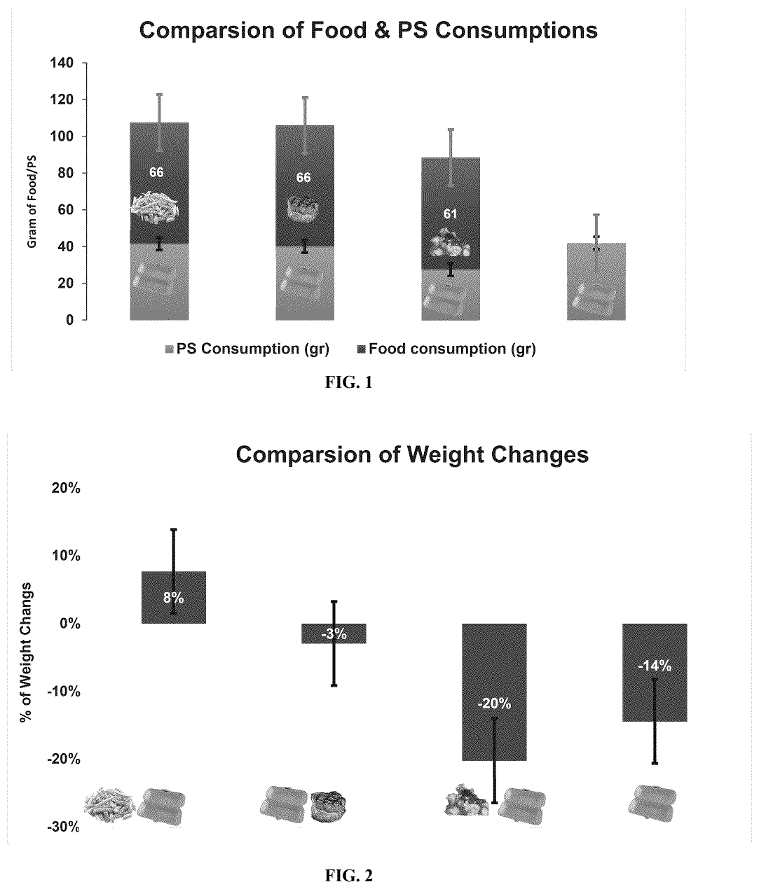

Consumption Rate. Superworm consumption of food and Polystyrene (PS) across four different groups is shown in FIG. 1. Food consumption is generally higher than PS consumption because the supermoms tend to finish the provided food first and then eat the PS. Carbohydrate and protein groups have the highest overall consumption. However, the food consumption is not significantly different between the three experimental groups.

Among three experimental groups, Superworms that received fatty food has significantly lower PS consumption. However, the PS consumption wasn't significantly different between the Faty food and the control group. Overall, providing food with PS did not have any significant impact on PS consumption. FIG. 1 shows a comparison of food and PS consumption between experimental and control groups.

Changes in Superworms' Weight. FIG. 2 shows the percentage of weight changes associated with different food items. According to these results, Superworm weight was increased by 8% in the group that received carbohydrates. This is very significant and important as usually superworm weight decreases over time. Consuming protein and fat are associated with significant weight loss, at 3% and 20% respectively. In addition, solely dieting on PS caused a 14% weight loss. FIG. 2 shows a comparison of Superworm changes between experimental and control groups.

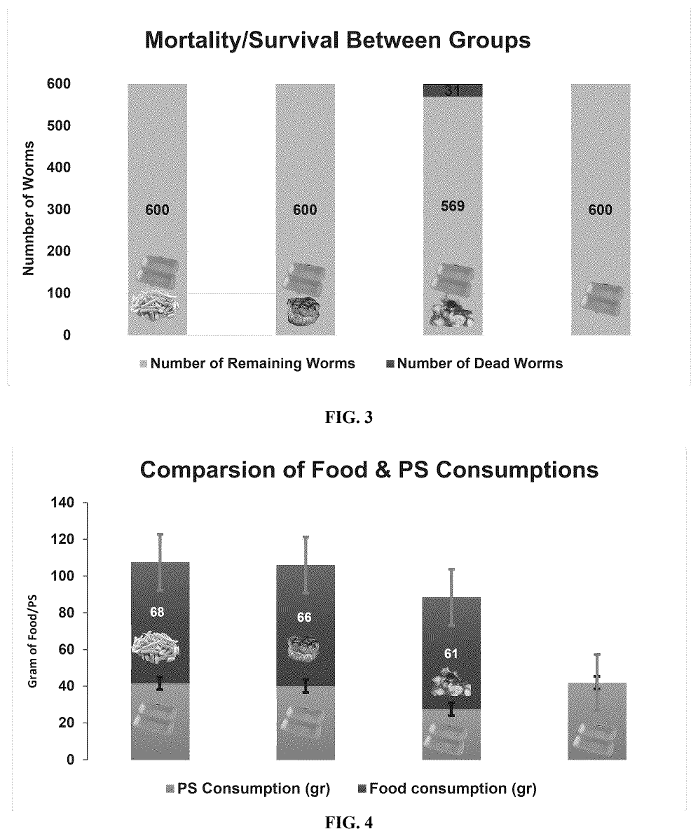

Mortality Rates. Superworm's survival rate is shown in FIG. 3. survival rate of 100% (no dead) was seen in the groups that received Carbohydrates, Protein and no food. Superworms that received the fatty food has 95% survival rate which was not significantly different than other groups. Overall, Superworms demonstrated remarkable resilience to shifts in their food source. FIG. 3 shows the survival/mortality between experimental and control groups.

### Example 2

A series of experiments were conducted utilizing the same packing material combined with leftover food, this time employing a different species, Tenebrio molitor (commonly known as the Mealworm). The results from these trials indicate that the Mealworm is also highly effective at biodegrading both the food packaging materials and the food. In certain experimental treatments, the Mealworms demonstrated superior performance compared to the Superworms, showing a species-specific advantages in biodegradation efficiency. Furthermore, an intriguing discovery emerged during these experiments: the adult Mealworm beetles exhibited the ability to biodegrade polystyrene (PS), a capability that was notably absent in the adult Superworm beetles, which showed no inclination to engage with this material. This finding highlights a significant difference in the ecological roles and degradation potentials between these two species with regard to waste management and material breakdown.

A commercially available population of mealworm larvae (Tenebrio molitor) was obtained from Carolina Biological Supply (Item #: 144264). Upon arrival, the mealworms were acclimated to the laboratory environment for two weeks to minimize stress-related impacts on the experiment. They were provided with a standardized diet of bran meals obtained from Carolina Biological Supply (Item #: 144320). For this study, Dart takeout containers (representing polystyrene) were used sourced from the UNT Dallas café, where other food items were obtained. Mealworms were housed in plastic containers with mesh screens as floors and without lids. These containers were maintained in a controlled laboratory environment at 25±1° C. and 65±5% relative humidity.

For the experiment, 2,400 mealworms were divided into three experimental groups and one control group. To account for potential variability, each group was subdivided into three replicate containers, with each container housing 200 mealworms (a total of 600 mealworms per group). This experimental design ensures sufficient statistical power. Each group received a weekly allotment of 3 g of food and 3 g of polystyrene. The first experimental group was provided with a baked potato to represent carbohydrate macromolecules. The second group received baked red meat to represent protein macromolecules. The third group was given baked fat trimmed from the meat to simulate lipid macromolecules. The control group received only polystyrene, with no additional food source.

Consumption Rate. Mealworms' consumption of food and Polystyrene (PS) across four different groups is shown in FIG. 4. Food consumption is generally higher than PS consumption because the mealworms tend to finish the provided food first and then eat the PS. Carbohydrate and protein groups have the highest overall consumption. However, the food consumption is not significantly different between the three experimental groups. FIG. 4 is a graph that shows a comparison of food and PS consumption between experimental and control groups.

Among three experimental groups, Mealworms that received fatty food has significantly lower PS consumption. However, the PS consumption wasn't significantly different between the Faty food and the control group. Overall, providing food with PS didn't have any significant impact on PS consumption.

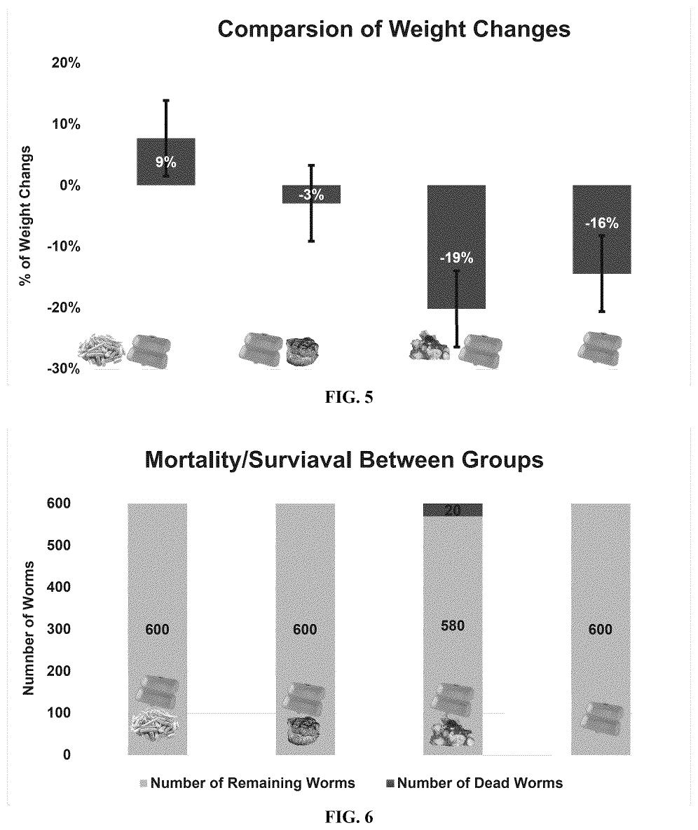

Changes in Mealworms'Weight. FIG. 5 shows the weight changes associated with different food items. According to these results, the mealworms'weight increased by 9% in the group that received carbohydrates. This increase is notable, as mealworm weight typically decreases over time. Consuming protein and fat was associated with significant weight loss of 3% and 19%, respectively. Additionally, a diet of only polystyrene resulted in a 16% weight loss. FIG. 5 is a graph that shows a comparison of Mealworm changes between experimental and control groups.

Mortality Rates. FIG. 6 shows the mealworms' survival rates. A survival rate of 100% (no deaths) was observed in the groups that received carbohydrates, protein, or only polystyrene. Mealworms that received fat trimmed from meat had a 97% survival rate, which was not significantly different from the other groups. Overall, mealworms demonstrated high resilience to changes in their food source. FIG. 6 is a graph that shows the survival/mortality between experimental and control groups.

It is contemplated that any aspects of the disclosure discussed in this specification can be implemented with respect to any method, kit, reagent, or composition of the disclosure, and vice versa. Furthermore, compositions of the disclosure can be used to achieve methods of the disclosure.

It will be understood that particular aspects described herein are shown by way of illustration and not as limitations of the disclosure. The principal features of this disclosure can be employed in various aspects without departing from the scope of the disclosure. Those skilled in the art will recognize, or be able to ascertain using no more than routine experimentation, numerous equivalents to the specific procedures described herein. Such equivalents are considered to be within the scope of this disclosure and are covered by the claims.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In aspects of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the disclosure(s) set out in any claims that may issue from this disclosure. Specifically, and by way of example, although the headings refer to a “Field of Invention,” such claims should not be limited by the language under this heading to describe the so-called technical field. Further, a description of technology in the “Background of the Invention” section is not to be construed as an admission that technology is prior art to any disclosure(s) in this disclosure. Neither is the “Summary” to be considered a characterization of the disclosure(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure but should not be constrained by the headings set forth herein.

All of the compositions and/or methods disclosed and claimed herein can be made and executed without undue experimentation in light of the present disclosure. While the compositions and methods of this disclosure have been described in terms of preferred aspects, it will be apparent to those of skill in the art that variations may be applied to the compositions and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### References

Chen, Z., Zhang, Y., Xing, R., Rensing, C., Lü, J., Chen, M., Zhong, S., & Zhou, S. (2023). Reactive Oxygen Species Triggered Oxidative Degradation of Polystyrene in the Gut of Superworms (Zophobas atratus Larvae). Environmental science & technology, 57(20), 7867-7874. doi.org/10.1021/acs.est.3c00591.
Hong Rae K., Hyun Min L., Hee Cheol Y., Eunbeen J., Sukkyoo L., Jiaojie L., Dae-Hwan K., (2020). Biodegradation of polystyrene by pseudomonas sp. isolated from the gut of superworms (larvae of zophobas atratus). Environmental science & technology. pubmed.ncbi.nlm.nih. gov/32374590/.
Rumbos, C. I., & Athanassiou, C. G. (2021). The superworm, Zophobas Morio (Coleoptera: Tenebrionidae): A “sleeping giant” in nutrient sources. OUP Academic. academic.oup.com/jinsectscience/article/21/2/13/6218202.
Sun, J., Prabhu, A., Aroney, S. T. N., & Rinke, C. (2022). Insights into plastic biodegradation: community composition and functional capabilities of the superworm (Zophobas morio) microbiome in styrofoam feeding trials. Microbial genomics, 8(6), mgen000842. doi.org/10.1099/mgen.0.000842.
Xu, L., Li, Z., Wang, L., Xu, Z., Zhang, S., & Zhang, Q. (2024). Progress in polystyrene biodegradation by insect gut microbiota. World journal of microbiology & biotechnology, 40(5), 143. doi.org/10.1007/s11274-024-03932-0.
Zielińska, E., Zieliński, D., Jakubczyk, A., Karaś, M., Pankiewicz, U., Flasz, B., Dziewięcka, M., & Lewicki, S. (2021). The impact of polystyrene consumption by edible insects Tenebrio molitor and Zophobas morio on their nutritional value, cytotoxicity, and oxidative stress parameters. Food Chemistry, 345, 128846. doi.org/10.1016/j.foodchem.2020.128846.

What is claimed is:

1. A method of degrading a combination of food stuffs and non-biodegradable food packaging, the method comprising:
obtaining a combination of the food stuffs and non-biodegradable food packaging;
contacting the combination of food stuffs and non-biodegradable food packaging with a superworm or a mealworm; and
incubating the combination of food stuffs and non-biodegradable food packaging and the superworm or mealworm under conditions in which the superworm or mealworm degrades both the food stuffs and non-biodegradable food packaging.

2. The method of claim 1, wherein the food stuffs and non-biodegradable food packaging are both carbon sources and the food stuffs comprise at least one of: plants, meats, and fats.

3. The method of claim 1, wherein the non-biodegradable food packaging is selected from at least one of: polystyrene (PS), polyethylene (PE), polypropylene (PP), polyvinyl chloride (PVC), polyethylene terephthalate (PET), or polycarbonate (PC).

4. The method of claim 1, wherein the superworm is Zophobas morio and the mealworm is Tenebrio molitor.

5. The method of claim 1, wherein the superworm or mealworm at least one of:
incubated with the food stuffs and non-biodegradable food packaging for a period of time that ranges from about 10, 15, 20, 25, 30, 40, 45, 50, 60, 70, 75, 80, 90, 100, 110, 120, 130, 140, or 150 days;
gains weight during the incubating step;
are grown with the food stuffs and non-biodegradable food packaging for a period of time that ranges from about 2 hours to about 480 hours, from about 2 hours to about 360 hours, from about 2 hours to about 240 hours, from about 2 hours to about 120 hours, from about 2 hours to about 96 hours, from about 2 hours to about 72 hours, from about 2 hours to about 48 hours, or from about 2 hours to about 24 hours;
are grown with the food stuffs and non-biodegradable food packaging at a temperature that ranges from about 15° C. to about 45° C.;
are grown with the food stuffs and non-biodegradable food packaging at a temperature that ranges from about 25° C. to about 28° C.;
are grown with the food stuffs and non-biodegradable food packaging at a moisture content that ranges from about 60% to about 99%; grown with the food stuffs and non-biodegradable food packaging a moisture content that ranges from about 80% to about 90%; or
convert the food stuffs and non-biodegradable food packaging to carbon dioxide (CO₂).

6. The method of claim 1, further comprising adding carbohydrate food stuffs to meat- or fat-based foods during the incubating step to increase at least one of a total degradation of the food stuffs and non-biodegradable food packaging, increase a rate of degradation of the food stuffs and non-biodegradable food packaging, increase superworm or mealworm size and/or weight, or decrease superworm or mealworm death.

7. The method of claim 1, wherein the mealworm is Tenebrio molitor and it biodegrades polystyrene.

8. A method of degrading a combination of food stuffs and non-biodegradable food packaging, the method comprising:
obtaining or growing superworms or mealworms and mixing with the food stuffs and non-biodegradable food packaging; and
incubating the combination of food stuffs and non-biodegradable food packaging and the superworms or mealworms, wherein the superworms or mealworms degrade both the food stuffs and non-biodegradable food packaging.

9. The method of claim 8, wherein the food stuffs and non-biodegradable food packaging are both carbon sources and food stuffs comprise at least one of: plants, meats, and fats.

10. The method of claim 8, wherein the non-biodegradable food packaging is selected from at least one of: polystyrene (PS), polyethylene (PE), polypropylene (PP), polyvinyl chloride (PVC), polyethylene terephthalate (PET), or polycarbonate (PC).

11. The method of claim 8, wherein the superworm is Zophobas morio and the mealworm is Tenebrio molitor.

12. The method of claim 8, wherein the superworm or mealworm at least one of:
incubated with the food stuffs and non-biodegradable food packaging for a period of time that ranges from about 10, 15, 20, 25, 30, 40, 45, 50, 60, 70, 75, 80, 90, 100, 110, 120, 130, 140, or 150 days;
gains weight during the incubating step;
are grown with the food stuffs and non-biodegradable food packaging for a period of time that ranges from about 2 hours to about 480 hours, from about 2 hours to about 360 hours, from about 2 hours to about 240 hours, from about 2 hours to about 120 hours, from about 2 hours to about 96 hours, from about 2 hours to about 72 hours, from about 2 hours to about 48 hours, or from about 2 hours to about 24 hours;
are grown with the food stuffs and non-biodegradable food packaging at a temperature that ranges from about 15° C. to about 45° C.;
are grown with the food stuffs and non-biodegradable food packaging at a temperature that ranges from about 25° C. to about 28° C.;
are grown with the food stuffs and non-biodegradable food packaging at a moisture content that ranges from about 60% to about 99%;
are grown with the food stuffs and non-biodegradable food packaging a moisture content that ranges from about 80% to about 90%; or
convert the food stuffs and non-biodegradable food packaging to carbon dioxide (CO₂).

13. The method of claim 8, further comprising adding carbohydrate food stuffs to meat- or fat-based foods during the incubating step to increase at least one of a total degradation of the food stuffs and non-biodegradable food packaging, increase a rate of degradation of the food stuffs and non-biodegradable food packaging, increase superworm or mealworm size and/or weight, or decrease superworm or mealworm death.

14. The method of claim 8, wherein the mealworm is Tenebrio molitor and it biodegrades polystyrene.

15. A method of degrading a combination of food stuffs and non-biodegradable food packaging, the method comprising:
obtaining a combination of the food stuffs and non-biodegradable food packaging;
contacting the combination of food stuffs and non-biodegradable food packaging with Tenebrio molitor; and
incubating the combination of food stuffs and non-biodegradable food packaging and the Tenebrio molitor under conditions in which the Tenebrio molitor degrades both the food stuffs and non-biodegradable food packaging.

16. The method of claim 15, wherein the food stuffs and non-biodegradable food packaging are both carbon sources and the food stuffs comprise at least one of: plants, meats, and fats.

17. The method of claim 15, wherein the non-biodegradable food packaging is selected from at least one of: polystyrene (PS), polyethylene (PE), polypropylene (PP), polyvinyl chloride (PVC), polyethylene terephthalate (PET), or polycarbonate (PC).

18. The method of claim 15, wherein the Tenebrio molitor at least one of:
incubated with the food stuffs and non-biodegradable food packaging for a period of time that ranges from about 10, 15, 20, 25, 30, 40, 45, 50, 60, 70, 75, 80, 90, 100, 110, 120, 130, 140, or 150 days;
gains weight during the incubating step;
grown with the food stuffs and non-biodegradable food packaging for a period of time that ranges from about 2 hours to about 480 hours, from about 2 hours to about 360 hours, from about 2 hours to about 240 hours, from about 2 hours to about 120 hours, from about 2 hours to about 96 hours, from about 2 hours to about 72 hours, from about 2 hours to about 48 hours, or from about 2 hours to about 24 hours;
grown with the food stuffs and non-biodegradable food packaging at a temperature that ranges from about 15° C. to about 45° C.; grown with the food stuffs and non-biodegradable food packaging at a temperature that ranges from about 25° C. to about 28° C.;
grown with the food stuffs and non-biodegradable food packaging at a moisture content that ranges from about 60% to about 99%; grown with the food stuffs and non-biodegradable food packaging a moisture content that ranges from about 80% to about 90%; or
convert the food stuffs and non-biodegradable food packaging to carbon dioxide (CO₂).

19. The method of claim 15, further comprising adding carbohydrate food stuffs to meat- or fat-based foods during the incubating step to increase at least one of a total degradation of the food stuffs and non-biodegradable food packaging, increase a rate of degradation of the food stuffs and non-biodegradable food packaging, increase Tenebrio molitor size and/or weight, or decrease Tenebrio molitor death.
